(19) United States
(12) Patent Application Publication
(10) Pub. No.: US 2002/0106347 A1
(43) Pub. Date: Aug. 8, 2002

(54) Cytokines and their use in treatment and/or prophylaxis of breast cancer

(76) Inventors: Andrea Margaret Douglas, East Bentleigh (AU); Colin Glenn Begley, Donvale (AU)

Correspondence Address: Scully, Scott, Murphy & Presser, 400 Garden City Plaza, Garden City, NY, 11530, US

(21) Appl. No.: 09/819,097

(22) Filed: Mar. 5, 2001

Related U.S. Application Data
(63) Continuation of application No. 09/051,939, filed on Oct. 16, 1998, now abandoned.
Application No. 09/051,939 is a continuation of application No. PCT/AU96/00676, filed on Oct. 25, 1996.

(30) Foreign Application Priority Data
Oct. 27, 1995 (AU) PN 6242
Oct. 27, 1995 (AU) PN 6243
Mar. 27, 1996 (AU) PN 8966
Jun. 7, 1996 (AU) PO 0364

(51) Int. Cl. A61K 38/20; A61K 38/19; A61K 33/24; A61K 31/704

(52) U.S. Cl. 424/85.1; 424/85.2; 514/34; 514/110; 514/283; 424/649

(57) ABSTRACT

The present invention relates generally to a method for the treatment or prophylaxis of animals including humans suffering from or predisposed to breast cancer or other related cancers which comprises the use of cytokines and/or functionally active derivatives, hybrids and/or analogs thereof and to pharmaceutical compositions comprising same as therapeutic agents. In particular, but not exclusively, the present invention is directed to the use of cytokines which are ligands of members of the haemopoietin receptor super family or their derivatives, hybrids or analogues as therapeutic agents. The present application also contemplates breast cancer therapies and methods of suppressing growth of normal breast cells or breast cancer cells by the use of one or more cytokines optionally in combination with other therapeutic agents as well as the use of agonists or antagonists of cytokine activity. Particularly preferred are oncostatin M (OSM) and leukemia inhibitory factor (LIF).

The present invention relates generally to a method for the treatment or prophylaxis of animals including humans suffering from or predisposed to breast cancer or other related cancers which comprises the use of cytokines and/or functionally active derivatives, hybrids and/or analogs thereof and to pharmaceutical compositions comprising same as therapeutic agents. In particular, but Dot exclusively, the present invention is directed to the use of cytokines which are ligands of members of the haemopoietin receptor super family or their derivatives, hybrids or analogues as therapeutic agents. The present invention also contemplates breast cancer therapies and methods of suppressing growth of normal breast cells or breast cancer cells by the use of one or more cytokines optionally in combination with other therapeutic agents as well as the use of agonists or antagonists of cytokine activity.

Bibliographic details of the publications numerically referred to in this specification are collected at the end of the description. Sequence Identity Numbers (SEQ ID NOs.) for the nucleotide and amino acid sequences referred to in the specification are defined following the bibliography.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

Breast cancer is the most common malignancy of females in Western cultures and affects 1 in 13 women in Australia. The risk of death is related to a number of prognostic factors, the most powerful being whether the axillary lymph nodes are involved. The relapse rate at 10 years has been found to be as high as 85% for women with Stage II disease who display involvement of four or more axillary lymph nodes (Antman, 1992). In premenopausal patients with more than 10 involved nodes given standard dose adjuvant chemotherapy cyclophosphamide (C) methotrexate (M) 5 fluroviracil (F) prednisolone (P), greater than 70% have disease recurrence within 5 years of diagnosis. When estrogen receptor (ER) status is included as a prognostic indicator, those patients who have an ER negative tumor with 4 or more nodes involved have risk of recurrence and death which approaches that for patients with 10 or more positive lymph nodes.

In patients with a large primary tumor (eg >5 cm) and positive nodes (Stage III disease) the 5-year relapse rate is between 65% and 79% (Nemoto et al., 1980; Valagussa et al., 1978; Fisher et al., 1969). In Halsteds original series only 2 of 44 (5%) women with supraclavicular node involvement were free of cancer at 5 years (Halsted, 1907). In women with either fixed axillary nodes, axillary nodes more than 2.5 cm in diameter, tumor fixed to the chest wall or skin ulceration, 5-year disease free survival ranges from only 5% to 38% (Haagensen, 1986). The addition of either CMF chemotherapy or anthracycline-containing chemotherapy to local therapy (surgery+/−radiation to the breast) of stage III breast cancer appears to improve 5-year relapse free survival from 26% to 40% (Balawadjer, 1983). Stage IV breast cancer (metastatic disease) is invariably fatal. Thus, this is a disease with a poor outlook and for which new therapeutic strategies are required.

Chemotherapy is currently the mainstay of systemic therapy for breast cancer. Both retrospective (Bonadonna et al., 1981; Hryniuk and Levine, 1986; Hryniuk and Bush, 1984) and prospective (Jones et al., 1987; Focan et al., 1993; Carmo-Pereira et al., 1987; Tannock et al., 1988; Neri et al., 1993; Wood et al., 1994) data demonstrate a dose-response relationship for cytotoxic drugs in breast cancer. On the whole, these clinical studies show that any significant reduction of chemotherapy below a certain critical dose results in a compromise of response rate or shortening of survival. What is not clear is whether the in vivo dose-response curve in breast cancer is linear, so that ever increasing doses of cytotoxic agents result in a greater chance of response, or whether a plateau is reached, above which only greater toxicity is observed. A hint is given that the former might apply by the activity reported in studies of growth factor supported, dose-intensive regimens (Bronchud et al., 1989; Ardizzoni et al., 1994; Lalisang et al., 1994; Hoekman et al., 1991; Ferguson et al., 1993; Scinto et al., 1995; Piccart et al., 1995) and high-dose chemotherapy with autologous progenitor cell rescue in patients with metastatic breast cancer (Eddy, 1992). These single arm studies produced response rates of 60% to 100% which compares favourably to the 25% to 50% response rates obtained using conventional dose chemotherapy (Jain et al., 1985; Rozencweig et al., 1984; Van Oosterrom, 1987; Marchner, 1994).

While high-dose chemotherapy in metastatic breast cancer produces impressive response rates, it does not appear to have impacted on survival (Eddy, 1992). However, it might be expected to be more affective if given to patients with early stage breast cancer and features that suggest a likelihood of recurrence. The use of this approach in high-risk stage II and III breast cancer has produced promising initial results. Peters et at. (1993) delivered 4 cycles of standard-dose chemotherapy followed by high-dose cyclophosphamide, 1,3-bis(2-chloroethyl)-1-nitrosourea (BCNU) and cisplatin to 85 patients with high-risk (>10 involved axillary lymph nodes) stage II and III breast cancer. At a median follow-up of 2.5 years, the relapse-free survival was 72% and overall survival 82% (Peters et al., 1993). However, this regimen was associated with a 12% treatment-related mortality rate and a high incidence of chronic pulmonary drug toxicity (Todd et al., 1993). Gianni et al. (1992) gave 48 patients with >10 involved axillary nodes growth factor supported high-dose sequential chemotherapy. This regimen included cyclophosphamide, vincristine and methotrexate, cisplatin then melphalan. At a median follow-up of 21 months, relapse-free and overall survival was 93% (Gianni et al., 1992).

The present inventors recently conducted a feasibility study of three cycles of high-dose epirubicin (200 mg/m₂) and cyclophosphamide (4 gm/m₂) with peripheral blood progenitor support in women with high risk breast cancer (Basser et al., 1995). Myelosuppression and acute-non-haematological toxicities were marked, but reversible. Given that repeated use of anthracylines is limited by a dose-dependent, irreversible cardiomyopathy, cardiac function of patients was monitored closely. The left ventricular ejection fraction fell by 15% from baseline in only 4 of 30 patients (13%) when measured at the completion of the third cycle of chemotherapy. No patients at any stage developed symptoms or signs of congestive heart failure.

Therefore, although chemotherapy is efficious in the treatment of breast cancer, it is associated with significant toxicities. These toxicities justify the development of new approaches in this disease and have provided the impetus for further research into such new approaches.

Breast cancer has been recognised as a hormone-responsive tumor for nearly a century. The recognition that growth stimulation occurs following the interaction of estrogen with its receptor led to the development of competitively binding anti-estrogens capable of inhibiting breast cancer growth (Li et al., 1992). In addition the presence of estrogen receptors in breast tumors predicts both patient prognosis, and response to hormonal therapy. Moreover treatment with anti-estrogens improves survival of these patients (Osborne et al., 1991; Early Breast Trial Collaborative Group, 1992). More recently, the focus has moved to the possible role of additional growth factors and their cell surface receptors in development and progression of breast cancer and the possibility these pathways might serve as potential targets for therapy.

The in-vitro growth of breast cells requires exogenous serum-derived factors for optimal growth. Isolation of protein fractions responsible for this activity resulted in the recognition of several families of growth factors, including epidermal growth factor (EGF), transforming growth factor (TGF), fibroblast growth factor (FGF), insulin and insulin-like growth factors (IGF's), that are important in the growth of these cells. Likewise surface receptors for these growth factors have been identified on breast cells and are found with variable frequency in primary human breast tumor samples (Dickson and Lippmann, 1992; Kacinski et al., 1991; Harris, 1994; Chrysagekos and Dickson, 1994).

A number of “haemopoietic” growth-factors have been characterized that display diverse functions on many different tissues. In some cases, these growth-factors can even display conflicting actions on different tissues. Thus, for example, Leukemia Inhibitory Factor (LIF) was named because of its ability to induce terminal differentiation in murine Ml leukemia cells. Paradoxically, however, LIF acts to inhibit differentiation on embryonic stem (ES) cells. Moreover, LIF is also active on many cell types including neurones, hepatocytes, osteoblasts, adipocytes and megakaryocytes. These activities of LIE are mediated via specific cell-surface receptors that are present on all these tissues (Hilton et al., 1991). A number of other molecules also display a broad-range of activities. These pleiotropic molecules include cytokines such as interleukin-6 (IL-6), oncostatin M (OSM), ciliary neurotrophic factor (CNTF) and interleukin-11 (IL-11).

In accordance with the present invention, it is proposed that certain cytokines are effective in treating breast cancer.

Accordingly, one embodiment of the present invention contemplates a method for the treatment or prophylaxis of breast cancer in an animal, which method comprises adminitering to said animal an effective amount of one or more cytokines or functional derivatives or agonists of said one or more cytokines for a time and under conditions sufficient to ameliorate the effects of or to delay onset of said cancer.

Another embodiment of the present invention provides a method for suppressing growth, proliferation or enhancing differentiation of normal breast cells or breast cell carcinomas from animals or immortalised animal breast cell lines by contacting said cells with an effective amount of one or more cytokines or functional derivatives or agonists of one or more cytokines for a time and under conditions sufficient to suppress growth, proliferation or enhancing differentiation of said cells.

In a particularly preferred embodiment the present invention contemplates a method of treating or prophylaxis of breast cancer in an animal including a human which method comprises administering to said animal an effective amount of a cytokine selected from OSM, LIF, IL-6, IL-11 and EGF and other members of the EGF family, or functional derivatives or agonists thereof optionally in association with one or more other cytokines or other therapeutic agents.

Another embodiment of the present invention contemplates a therapeutic composition for the treatment of animals including humans suffering from breast cancer or having a predisposition to develop breast cancer which comprises one or more cytokines or functional derivatives or agonists thereof optionally in association with other therapeutic agents and also in association with one or more pharmaceutically acceptable carriers and/or diluents.

Administration of the active components of the present invention is for a time and under conditions sufficient for said components to exhibit the requisite effect. The term “breast cancer” is used in its broadest sense and includes all forms of breast cancer including but not limited to metastic breast cancer and early breast cancer. It also includes other cancers epidemiologically related to breast cancer.

By the term “animal” it is to be understood that the methods of treatment of the present invention are applicable to the treatment of breast cancer in all mammals and in particular humans as well as in livestock animals (e.g. sheep, cows, pigs, goats, horses, donkeys), laboratory test animals (e.g. mice, rats, guinea pigs, hamsters, rabbits), domestic companion animals (e.g. dogs, cats) and captive wild or tamed animals (e.g. monkeys, foxes, kangaroos, dingoes).

Preferably, the cytokine is a recombinant cytokine of human, murine, livestock animal, companion animal, laboratory test animal or captive wild animal origin. More preferably however, the cytokine is of human origin. The present invention extends to all cytokines which bind to surface receptors of breast cells whether they be normal breast cells, breast cell carcinomas or immortalised breast cell lines of human or animal origins, and which exhibit an activity on cell growth, proliferation or differentiation. In particular, the cytokines of the present invention include oncostatin M (OSM), interleukin-6 (IL-6), interleukin-11 (IL-11), leukemia inhibitory factor (LIF) and EGF and other members of the EGF family. Most preferably, the cytokine of the present invention is OSM of either human or murine origin, but preferably of human origin. In this regard, homologous or heterologous treatments are contemplated by the present invention. A homologous treatment employs a cytokine from one animal species in the treatment of an animal from the same species (e.g. human OSM in humans). Heterologous treatment employs a cytokine from one animal species in the treatment of an animal of a different species (e.g. murine OSM in humans).

The term “derivatives” extends to functionally active parts, mutants, fragments and analogues of cytokines which exhibit the desired activity herein described.

The terms “agonists” and “antagonists” are envisaged compounds which may or may not be cytokines but which facilitate cytokine interaction with its receptors on breast cells or breast cancer cells to ellicit an activity, preferably an enhanced or diminished activity depending on whether it is an agonist or antagonist, respectively. One example of an antagonist of a cytokine is use of antisense oligonucleotide sequences. Useful oligonucleotides are those which have a nucleotide sequence complementary to at least a portion of the protein coding or “sense” sequence which encodes the particular cytokine concerned can be utilised. These anti-sense nucleotides can be used to effect the specific inhibition of gene expression (Markus-Sekura, 1988). The antisense approach can cause inhibition of gene expression apparently by forming an anti parallel duplex by complementary base pairing between the antisense construct and the targeted mRNA, presumably resulting in hybridisation arrest of translation.

There have been several reports of antisense effects on cytokine-responsive cells, such as IL-β-responsive lymphokine-activated cells (Fujiwara and Grimm, 1992), TNF-α-responsive differentiating macrophages (Witsell and Schook, 1992), M-CSF-responsive HL-60 cells (Wu et al, 1990) and IL-6 responsive cells (Levy et al, 1991). These studies have demonstrated the critical role of these genes in the growth of different cell types.

The present invention extends to analogues of cytokines and their use in the treatment or prophylaxis of breast cancer. Analogues of cytokines contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succimic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids A list of unnatural amino acid, contemplated herein is shown in Table 1.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

These types of modifications may be important to stabilise the cytokines if administered to an individual or for use as a diagnostic reagent.

TABLE I

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | | Chexa L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycjne | Mtbug |
| L-α-methylcystejne | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

The cytokines may optionally be administered together with one or more other therapeutic agents. These other agents contemplated by the present invention are agents such as chemotherapeutic and hormonal agents which are well known in the art. Examples of some chemotherapeutic agents are cyclophosphamide, vincristine and methotrexate, cisplatin, melphalan and an example of a common hormonal type agent is tamoxifen. This list of other therapeutic agents is by no means exhaustive. Other useful molecules contemplated herein include taxol (and related molecules such as taxitere) and adriamycin. Such combination therapy may prove effective in treating metastatic breast cancer or in treatment of early breast cancer in particular.

It is contemplated by the invention that when one or more cytokines are administered in combination with other agents that the administration is done simultaneously or sequentially. Simultaneous administration occurs when the cytokine is co-administered with the other therapeutic agent. Sequential therapy includes a time difference between administration of the various molecules which may be in the order of seconds, minutes, hours, days, weeks or months depending upon the severity of the patient's condition, the type of mammal being treated and the effectiveness of the overall treatment.

It is also within the scope of the invention to administer a combination of different cytokines. Preferably, the combination comprises a haemopoetin receptor cytokine with another cytokine of the same family or comprises a haemopoietic receptor cytokine and a cytokine from another family. Preferably, the combination comprises OSM and at least one other cytokine. Even more preferably, the combination comprises OSM together with one or more of IL-6, IL-11, LIF and/or EGF or another member of the EGF family. For example, administration of OSM and IL-11 or OSM and LIF or OSM and IL-6 or OSM and EGF or OSM together with LIF, two or more of IL-6, IL-11 and EGF is clearly contemplated by the present invention.

The amount of cytokine administered is to be determined on a case by case basis taking into account the condition of the patient, species, weight, age, other concurrent treatments and other factors which would be apparent to a physician. As an example it is envisaged that an amount of from about 0.5 micrograms to about 2 milligrams of cytokine per kilogram of body weight per day may be administered. Naturally, dosage regimes may be adjusted to provide the optimum prophylactic or therapeutic response. For example, several divided dosages may be administered daily or the dose may be proportionally reduced as indicated by the particular therapeutic situation. Furthermore, lower amounts may be given but more frequently such as 0.1 to 10 μg per kilogram of body weight per day. Alternatively, larger amounts may be given but less frequently such as from 1 milligram to about 25 milligrams per kilogram of body weight per day.

A decided practical advantage is that the active compound may be administered in a convenient manner such as by the oral, intravenous (where water soluble), intramuscular, subcutaneously, intranasal, intradermal or suppository routes. The active compound may also be administered locally such as directly into tissue or via a slow release formulation. Depending upon the route of administration, the active ingredients may be required to be coated in a material to protect the ingredients from action of enzymes, acids and other natural conditons which may inactivate the ingredients. In order to administer cytokines by other than parenteral administration, they may be coated by or administered with, a material to prevent inactivation. For example, cytokines or in particular OSM, may be administered in an adjuvant formulation or co-administered with enzyme inhibitors or in liposomes.

Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water cytokine emulsions as well as conventional liposomes.

Cytokines may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microrganisms such as bacteria or fungi. The carrier can be a coolant of dispersion medium containing, for example, water, ethanol, polyol (for example, glycerol, propylene glycol, and liquid polyethylene glycol, and the like), suitable mixtures thereof and vegetable oils. The proper fluidity can be maintained, for example, by the use of a coating such as lecithin, by the maintenance of the required particle size in the case of dispersion and by the use of superfactants. The prevention of the action of microrganisms can be brought about by various antibacterial and anti fungal agents, for example, parabens, chlorobutanol, phenol, sorbic acid, thiomerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared incorporating the various sterilised active ingredient(s) into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile filtered solution thereof.

When the active ingredients are suitably protected as described above, the composition may be orally administered, for example, with an inert diluent or with an assimilable edible carrier, or it may be enclosed in hard or soft shell gelatine capsule, or it may be compressed into tablets, or it may be incorporated directly with the food of the diet. For oral therapeutic administration, the active compound may be incorporated with excipients and used in the form of ingestible tablets, buccal tables, troches, capsules, elixirs, suspension, syrups, waffers, and the like. Such compositions and preparations should contain at least 1% on weight of active compound. The percentage of the compositions and preparations may of course be varied and may conventionally be between about 5 to about 80% of the weight of the unit. The amount of active compound(s) in the pharmaceutical compositions is such that a suitable dosage will be obtained. Preferred compositions or preparations according to the present invention are prepared, so that an oral dosage unit form contains between about 0.5 nanogram and 320 milligram of active compound.

The tablets, troches, pills capsules and the like may also contain the following: a binder such as gum gragacanth, acacia, corn starch or gelatin; excipients such as dicalcium phosphate, a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained release preparations and formulations.

As used herein “pharmaceutically acceptable carriers and/or diluents” include any and all solvents, dispersion media, aqueous solutions, coatings, antibacterial and antifungal agents isotonic and absorption delaying agents and the like. The use of such media and agents for pharmaceutical active substance is well known in the art. Except insofar as any convential media or agent is incompatible with the active ingredient, use thereof in the pharmaceutics compositions is contemplated. Supplementary active ingredients can also be incorporated into the compositions.

The preferred cytokine for practice of the present invention is OSM. The OSM employed is preferably as described in U.S. Pat. No. 5,428,012. Preferably, the OSM comprises an amino acid sequence as set forth in FIG. 3 in U.S. Pat. No. 5,428,012 or is similar thereto or is a derivative or agonist thereof. In its most preferred form, the OSM comprises an amino acid sequence which has at least 40%, more preferably at least 50%, even more preferably at least 60%, still more preferably at least 70-80% and yet even more preferably at least 90-95%, similarity or identity to one or more regions of the amino acid sequence set forth in FIG. 3 of U.S. Pat. No. 5, 428,012. The cytokine may also contain single or multiple amino acid insertions, deletions and/or additions to the naturally occurring sequence and may be derivatised or fragmented to a part carrying the active site of the cytokine. All such derivatives or fragmented cytokine molecules are encompassed by the present invention and are included in the expression “cytokine”, provided all such molecules have the effect of altering and preferably reducing growth, proliferation or promoting differentation of breast cancer cells.

Administration may be by any suitable route such as intravenous, intranasal, subcutaneous, intraperitoneal, intramuscular, intradermal, infusion, suppository, implant and oral including slow release capsules. Where cytokines may have a relatively short serum half life, the injected preparation may need to be modified to reduce serum degradation and/or alternative routes of administration employed. Administration may also be by gene therapy including expression of the particular cytokine gene in vectors which are introduced to the mammal to be treated. Alternatively, the cytokine gene can be expressed in bacteria which are then incorporated into the normal flora of the host.

The effective amount of cytokine and particularly OSM will depend on the animal and the condition to be treated. For example, amounts ranging from about 0.1 ng/kg/body weight/day to about 1000 μg/kg/body weight/day are contemplated to be useful in breast cancer therapy. More preferably, the effective amount is 1 ng/1 kg body weight/day to 100 μg/kg body weight/day. Even more preferably, the effective amount is 10 ng/kg body weight/day to 10 μg/kg body weight/day. Such effective amounts may reflect actual administration protocols or may reflect an average of an alternate administration protocol. The protocol may be varied to administer cytokine or particularly OSM per hour, week or month or in conjunction with other therapeutic agents.

In all of the above cases, the present invention also extends to the use of derivatives of cytokines. By derivatives is meant recombinant, chemical or other synthetic forms of OSM or other cytokines and/or any alteration such as addition, substitution and/or deletion to the amino acid sequence component of the molecule or to the carbohydrate or other associated molecule moiety of OSM or other cytokine provided the derivative possesses the ability to alter and particularly to slow growth, proliferation or enhancing differentiation of breast cells. Accordingly, reference herein to OSM or to a cytokine includes reference to its derivatives.

The present invention is further described by reference to the following non-limiting Figures and/or Examples.

In the Figures:

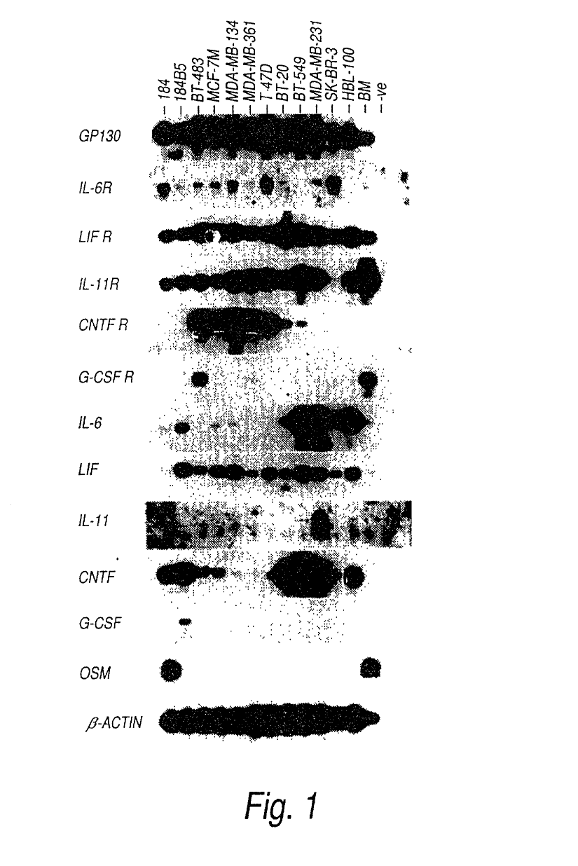

FIG. 1 is a photographic representation of an analysis of growth factor/receptor expression in breast cancer cells assessed by reverse-transcriptase polymerase chain reaction, RT-PCR. Autoradiograph of RT-PCR products obtained from the analysis of breast cell mRNA. Products were transferred to nylon membranes prior to being probed with a ³²P-labelled oligonucleotide corresponding to the respective growth factor/receptor (Table 1). Lanes 1-12 contain DNA samples from the following cell lines respectively, 184, 184B5, BT-483, MCF-7M, MDA-MB-134, MDA-MB-361, T-47D, BT-20, BT-549, MDA-MB-231, SK-BR-3 and HBL-100. Samples in lanes 1 & 2 are from normal breast epithelial cell lines, lanes 3-7 are from estrogen receptor (ER) positive breast cancer cell lines, and lanes 8-12 are from ER negative breast cancer cell lines. Lane 13 is a positive control containing DNA from bone marrow and lane 14 is a negative control in which DNA was ommitted from the PCR. Rows A-K represent products detected when the membranes were hybridised to oligonucleotides specific for the following growth factors/receptors respectively, gp130, IL-6R, LIFR, IL-11R, CNTFR, G-CSFR, IL-6, LIF, IL-11, CNTF, G-CSF, OSM and β-ACTIN. Control samples, obtained when reverse transcriptase was ommitted from the initial cDNA synthesis of each sample, gave no detected signal.

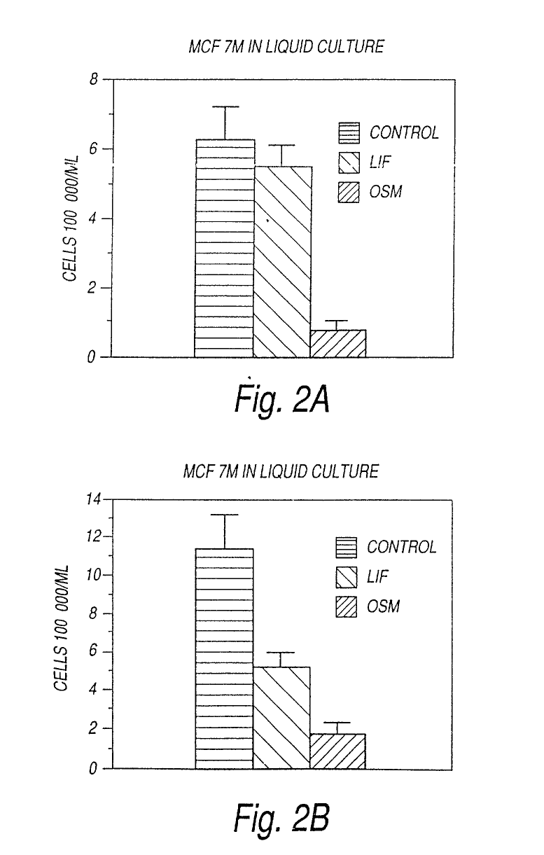

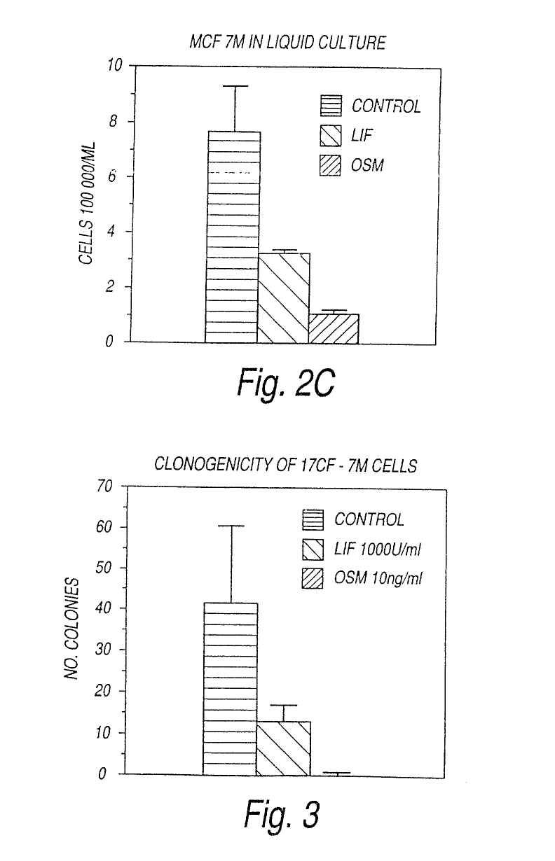

FIGS. 2A, B and C are graphical representations of MCF-7M cells in liquid culture. 10⁴ MCF-7M cells were cultured in 500 μl RPMI/10% bovine calf serum (BCS) (v/v) containing the indicated concentration of each of the growth factors (OSM or LIF). After 7 days culture viable cells were counted using a hemacytometer. Cell numbers reported here are the result of one experiment, each performed in triplicate.

FIG. 3 is a graphical representation showing clonogenicity of MCF-7M cells. Following suspension culture MCF-7M cell viability was measured by agar culture. Cells were plated in agar with the indicated concentrations of each of the growth factors. After 14 days culture colonies of cells were counted. Cell numbers reported here are the results of 3 experiments, each performed in triplicate, and are expressed according to the number of cells that went into suspension culture.

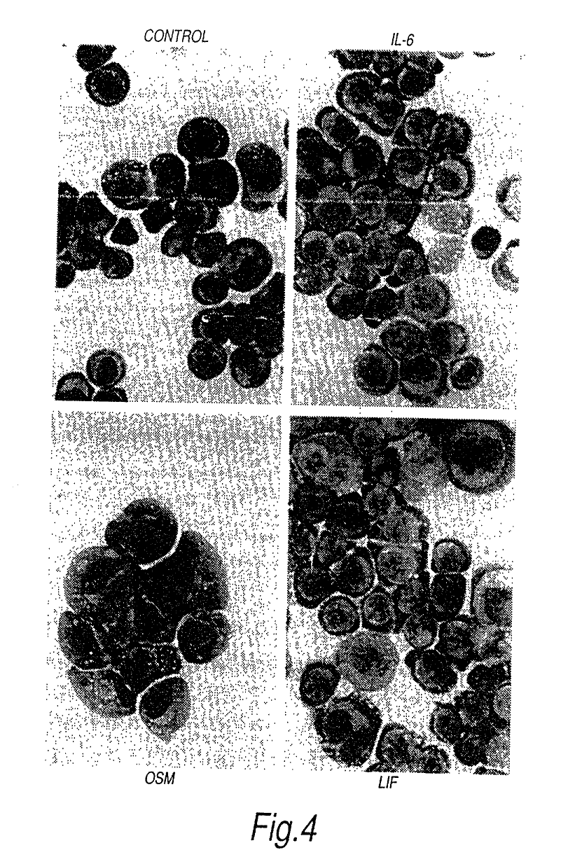

FIG. 4 is a photographic representation showing morphology of growth factor stimulated MCF-7M cells. Following 1 week in suspension culture containing the various growth factors, MCF-7M cells were cytospun onto slides and subsequently stained with Giemsa.

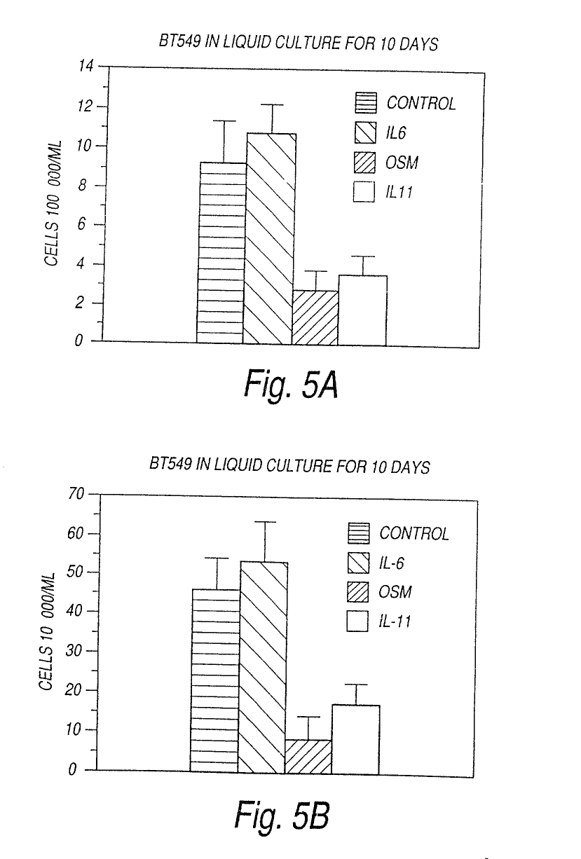

FIGS 5A and B are graphical representations of BT-549 cells in liquid culture. 10⁴ BT-549 cells were cultured in 500 μl RPMI/10% (v/v) BCS containing the relevant concentration of each of the growth factors (OSM, IL-11 and IL-6). After 10 days, culture viable cells were counted using a hemacytometer. Cell numbers reported here in the two graphs are the results of 1 experiment performed in triplicate.

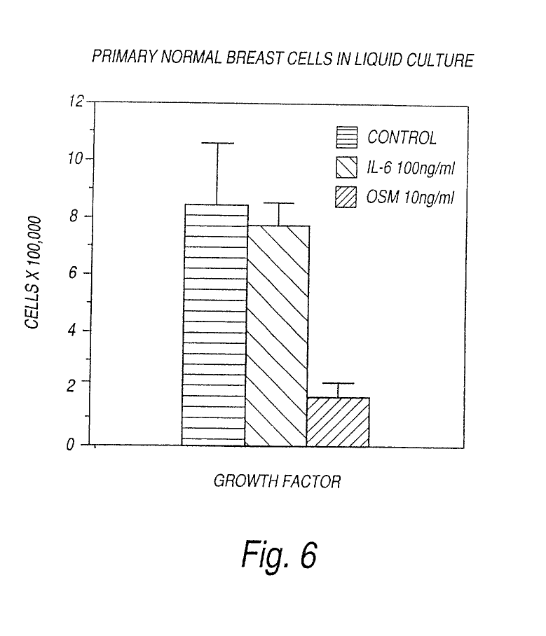

FIG. 6 is a graphical representation of primary normal breast cells in suspension culture. 10⁴ primary normal breast cells were cultured in 500 μl serum free breast media containing the indicated concentration of each of the growth factors (OSM and 116). After 7 days culture viable cells were counted using a hemacytometer. Cell numbers reported here are the results of 1 experiment, performed in triplicate.

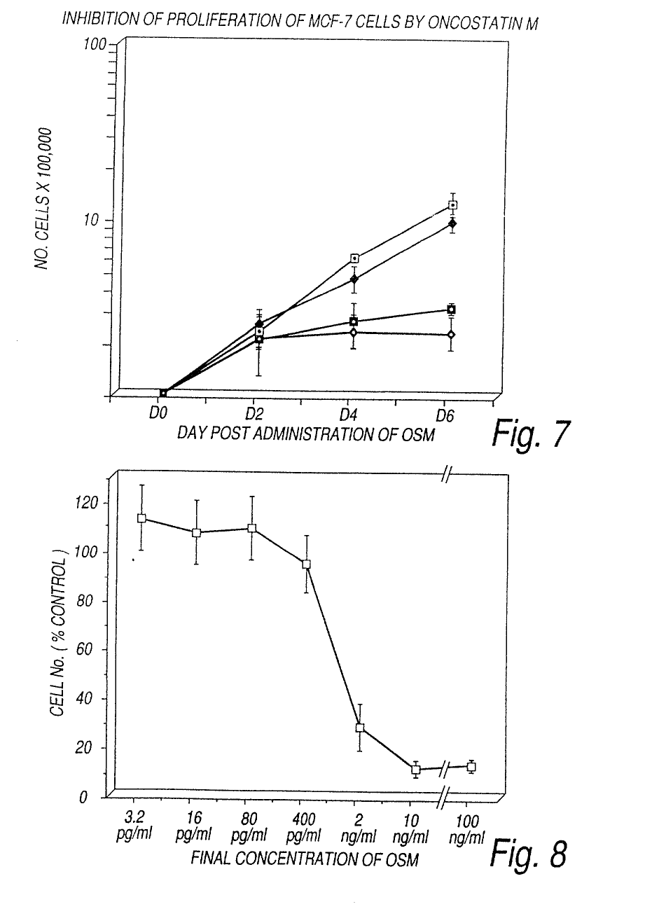

FIG. 7 is a graphical representation showing inhibition of proliferation of MCF-7 cells by Oncostatin M (OSM). 10⁴ MCF-7 cells were cultured in 500 μl RPMI/10% (v/v) BCS with the indicated concentrations of OSM. At 2, 4 and 6 days, viable cells were counted using a hemacytometer. Results are from 3 experiments, each performed in triplicate.

FIG. 8 is a graphical representation showing that MCF-7 cells are inhibited in a dose-dependent fashion by Oncostatin M (OSM). 10⁴ MCF-7 cells were cultured in 500 μl RPMI/10% (v/v) BCS with the indicated concentrations of OSM. After 7 days viable cells were counted, and cell number expressed as a percentage of the corresponding untreated control value.

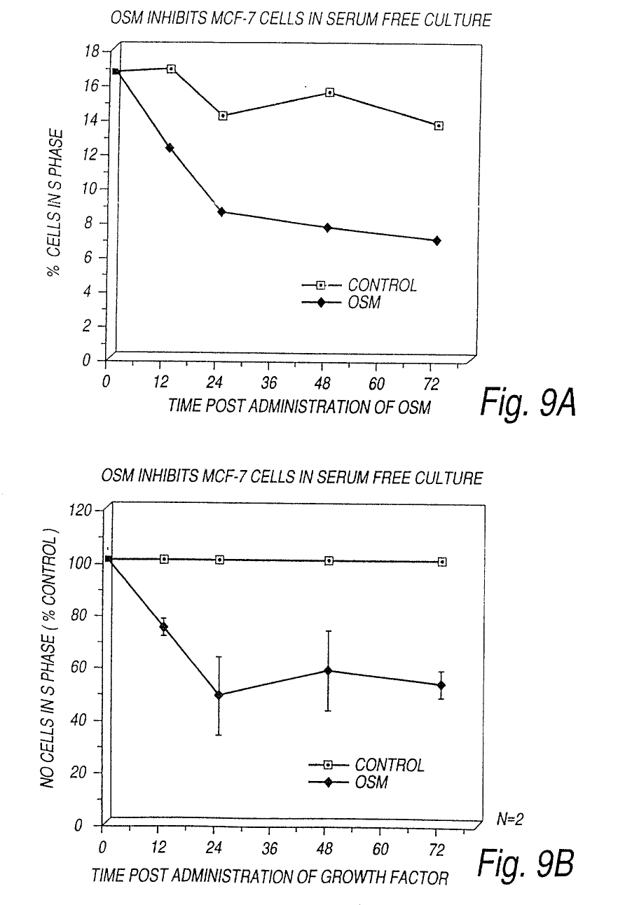

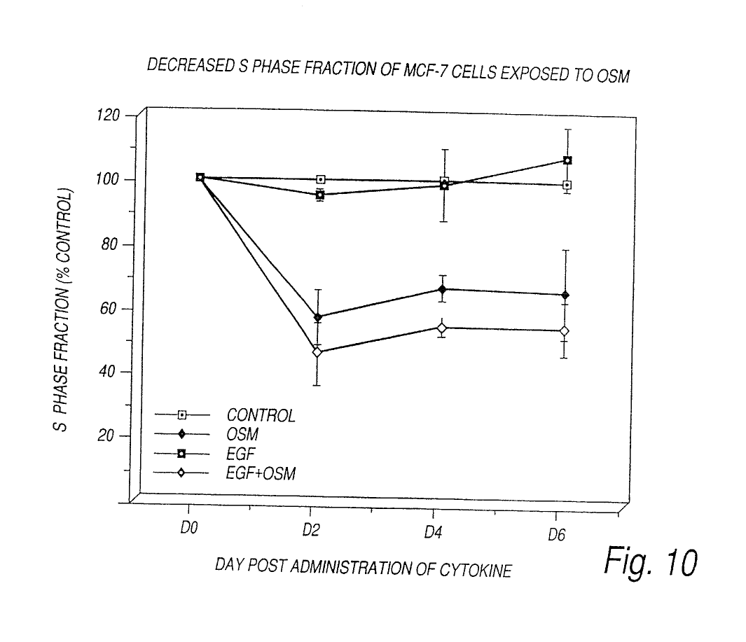

FIG. 9 is a graphical representation showing the effect of OSM on cell cycle. MCF-7 cells treated with OSM while growing in serum-free medium were harvested by treatment with trypsin and stained for DNA content analysis by flow cytometry. Cell cycle distributions were calculated by computer fitting of the resultant histograms. FIG. 10(a) represents a typical experiment indicating that the percentage of cells in S phase following treatment with OSM decreases from approximately 15% to 8% over a 72 hour time period. FIG. 10(b) represents data from 2 experiments (performed in triplicate) where the number of cells in S phase are represented as a percentage of the corresponding untreated control value.

FIG. 10 is a graphical representation showing the effect of EGF and OSM on cell cycle. MCF-7 cells treated with OSM, Epidermal Growth Factor (EGF) or both OSM and EGF while growing in serum-free medium were harvested by treatment with trypsin and stained for DNA content analysis by flow cytometry. Cell cycle distributions were calculated by computer fitting of the resultant histograms. Results represent the combined data from 3 experiments (performed in triplicate) where the number of cells in S phase are represented as a percentage of the corresponding untreated control value.

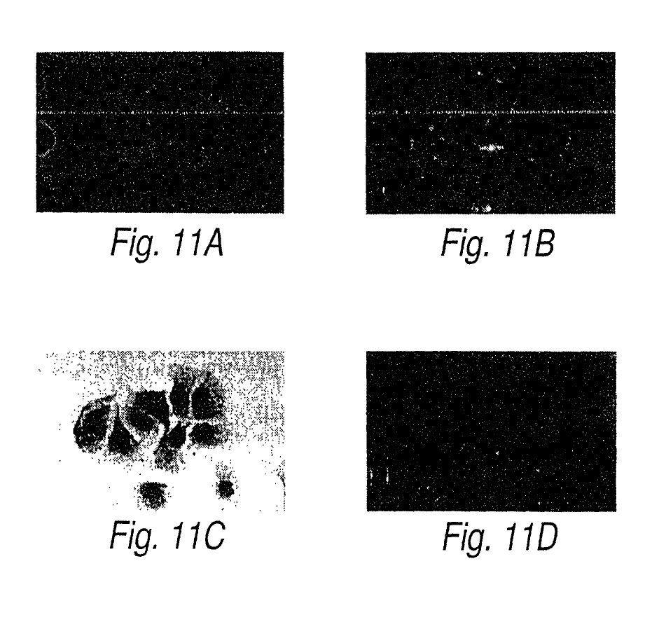

FIG. 11 is a photographic representation showing cell morphology after exposure to OSM. MCF-7 cells from control cultures and appears of cells after 7 days in OSM. A) Control cells, 10× magnification. B) OSM treated cells, 10× magnification. C) OSM treated cells, 40× magnification. D) OSM treated cells, 100× magnification.

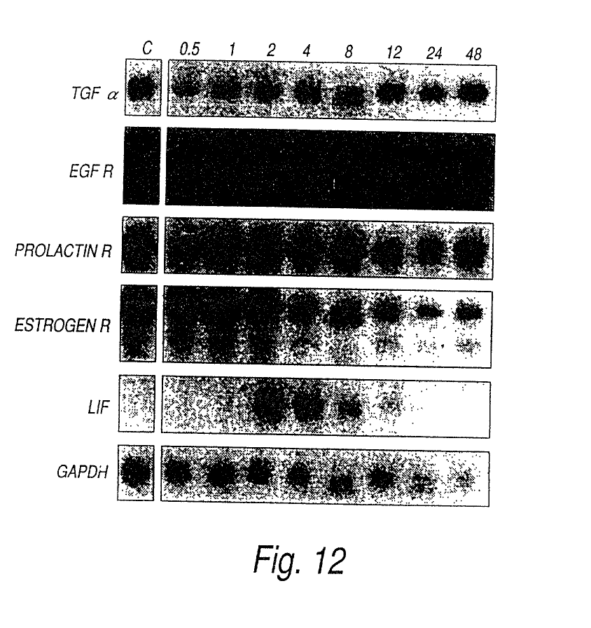

FIG. 12 is a photographic representation showing effect of OSM on the expression of Transforming Growth Factor α (TGFα), Epidemal Growth Factor Receptor (EGFR), Prolactin Receptor (PRLR), Estrogen Receptor (ER) and LIF mRNA. Cells growing in the presence of 10% (v/v) BCS were treated with OSM (10 ng/ml) and at the indicated time points duplicate 150 cm² flasks were harvested and mRNA extracted for Northern analysis. Results for control cells (C) are also shown. The same filter has been probed successively with a ³²P-labelled cDNA corresponding to each mRNA species. mRNA loading was evaluated by reprobing the filter with a fragment complementary to GAPDH. mRNA species of the following sizes were obtained: TGFα, 4.8 kb; PRLR, 10.5 and 8.6 kb; EGFR, 10.5 and 5.8 kb; ER, 6.5 and 3.8 kb and LIF, approx. 4.8 kb.

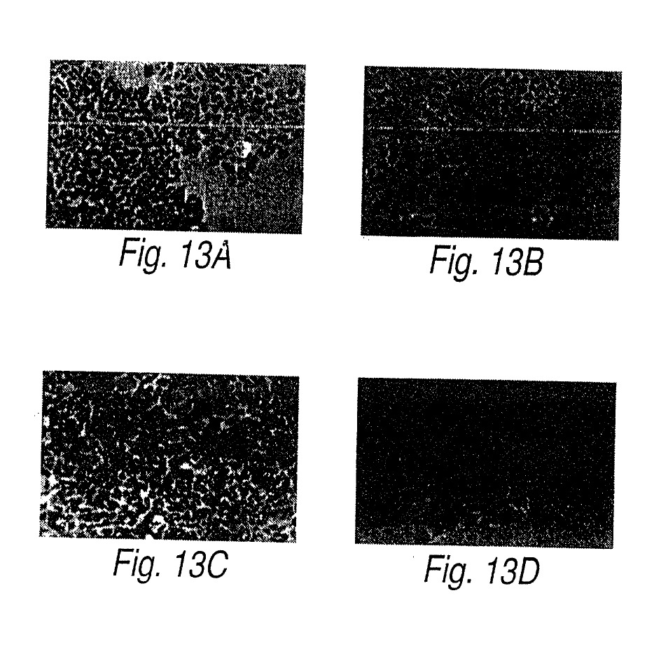

FIG. 13 is a photographic representation showing ER expression in OSM and EGF treated cells. MCF-7 cells were grown on chamber slides for 6 days in RPMI/10% (v/v) BCS with the following growth factors, prior to being stained with an antibody specific for ER. A) Control, B) OSM, C) EGF, D) OSM and EGF. Cells stained brown indicate ER positivity.

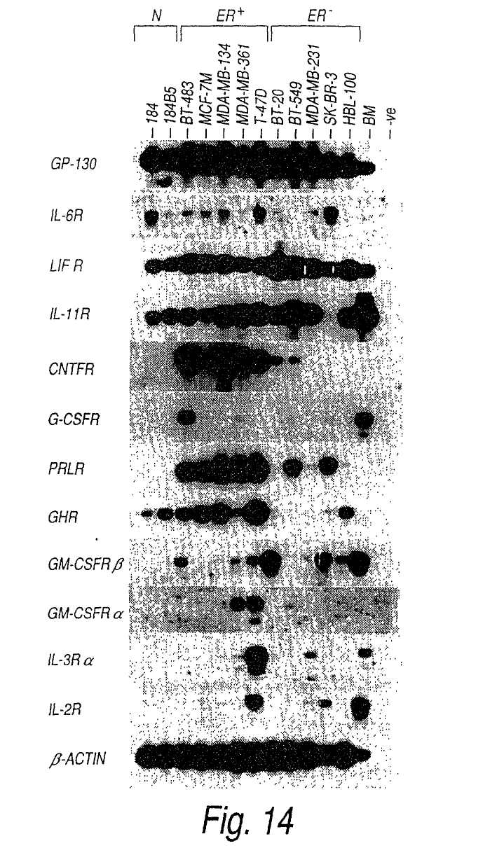

FIG. 14 is a photographic analysis of growth factor receptor expression in breast cancer cell lines assessed by RT-PCR Autoradiograph of RT-PCR products obtained from the analysis of breast cell mRNA. Samples in lanes 1 & 2 are from normal breast epithelial cell lines, lanes 3-7 are from ER positive breast cancer cell lines, and lanes 8-12 are from ER negative breast cancer cell lines. Lane 13 is a positive control containing RNA from bone marrow (BM) and lane 14 is a negative control in which RNA was ommitted from the PCR (-ve). Products were transferred to nylon membranes prior to being probed with a “³²P-labelled oligonucleotide corresponding to the receptor indicated on the left and β-Actin as a control. Lanes 1-12 contain RNA samples from the following cell lines respectively, 184, 184B5, BT-483, MCF-7M, MDA-MB-134, MDA-MB-361, T-47D, BT-20, BT-549, MDA-MB-231, SK-BR-3 and HBL-100. Control samples, obtained when reverse transcriptase was ommitted from the initial cDNA synthesis of each sample, gave no signal.

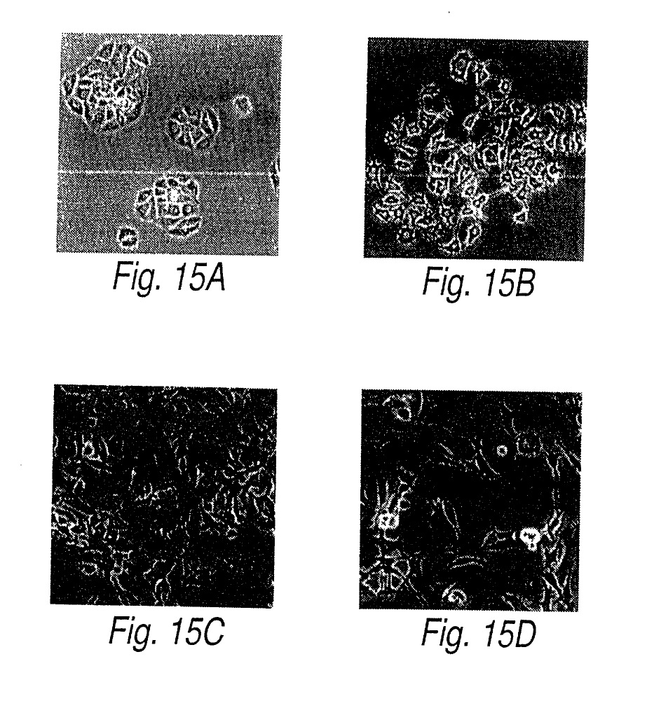

FIG. 15 is a photographic representation showing cell morphology after exposure to OSM. MCF-7 cells from control cultures (Panel A) and appearance of cells after culture for 14 days in OSM (10 ng/ml) (Panel B). MDA-MB-231 cells from control cultures (Panel C) and appearance of cells after culture for 7 days in OSM (Panel D).

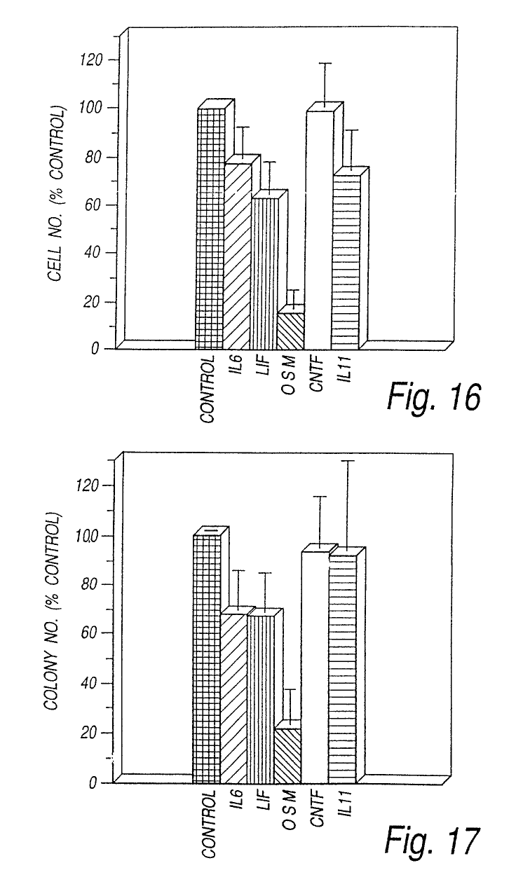

FIG. 16 is a graphical representation showing inhibition of MCF-7 cells after 7 days in suspension culture. 10⁴MCF-7 cells were cultured in 500 μl RPMI/10% (v/v) BCS with the indicated growth factor. After 7 days viable cells were counted using a hemacytometer. Results are from 9 experiments, each performed in triplicate.

FIG. 17 is a graphical representation showing clonogenicity of MCF-7 cells after 1 week in suspension culture. Following suspension culture clonogenicity of MCF-7 cells was assayed in agar culture. Cells were plated in agar with the indicated growth factor and maintained at 37° C0 in a humidified incubator with 5% CO₂ in air. After 14 days colonies of cells were counted. Results are from 9 experiments using IL-6, LIF and OSM, and 5 experiments using CNTF and IL-11. Each experiment was performed in triplicate, and colony numbers are expressed as a percentage of untreated controls.

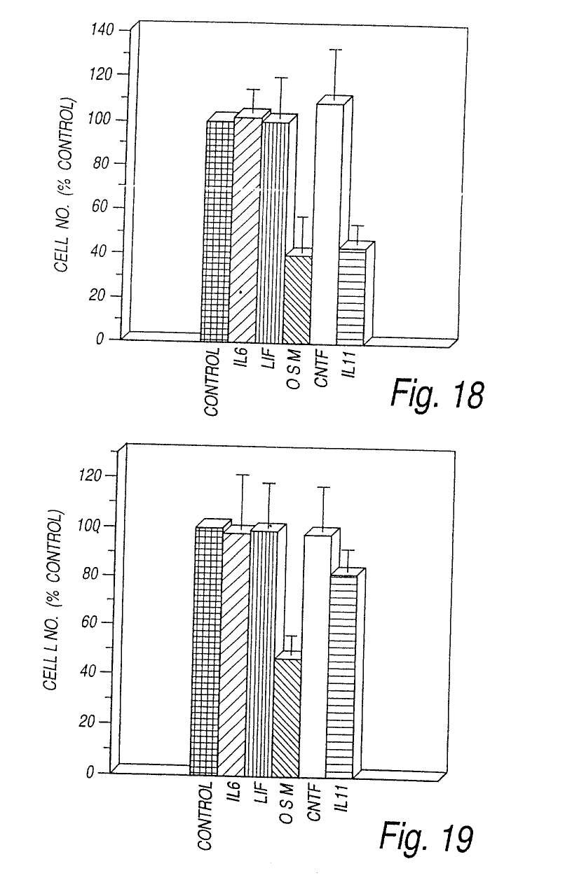

FIG. 18 is a graphical representation showing BT-549 cells after 10 days in suspension culture. 10⁴BT-549 cells were cultured in 500 μl RPMI/10% (v/v) BCS with the indicated growth factor. After 10 days culture viable cells were counted using a hemacytometer. Results are from 6 experiments, each performed in triplicate.

FIG. 19 is a graphical representation showing MDA-MB-231 cells after 7 days in suspension culture. 10⁴MDA-MB-231 cells were cultured in 500 μl RPMI/10% (v/v) BCS with the indicated growth factor. After 7 days viable cells were counted using a hemacytometer. Results are from 8 experiments, each performed in triplicate.

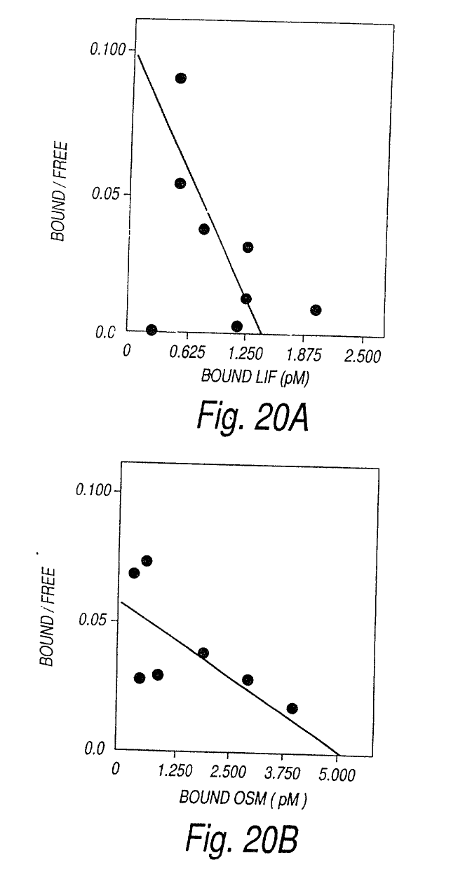

FIG. 20 is a graphical representation showing Scatchard analyses of the saturation isotherms of LIF and OSM binding to breast cancer cell lines. Cells were incubated with various concentrations of labelled or unlabelled ligand in the presence or absence of a 10-100 fold excess of unlabelled ligand. After 18 hr on ice, bound and free ligand were separated by centrifugation through bovine calf serum. Bound and free ¹²⁵I-ligand was quantitated in a γ-counter and the data was depicted as a Scatchard transformation. Data was normalised for cell number and is shown as binding to 10⁶ cells. A) Saturation isotherm of LIF binding to the MCF-7 cell line. This analysis indicates high affinity binding of LIF with a dissociation constant of 14.6 pM and an estimated 57 receptors per cell. B) Saturation isotherm of OSM binding to the MDA-MB-231 cell line. This analysis indicates high affinity binding of OSM with a dissociation constant of 92 pM and an estimated 124 receptors per cell.

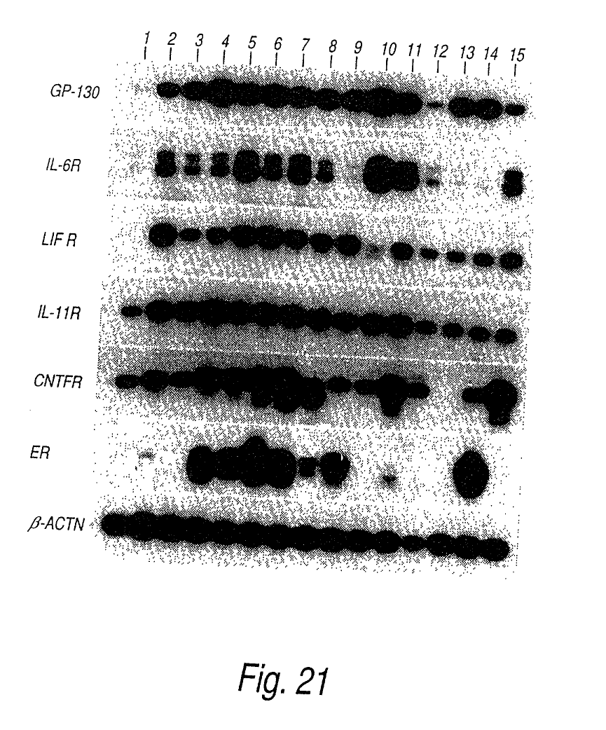

FIG. 21 is a photographic analysis of growth factor receptor expression in primary breast cancer tissue assessed by RT-PCR Autoradiograph of RT-PCR products obtained from the analysis of fresh breast tissue mRNA. Products were transferred to nylon membranes prior to being probed with a ³²P-labelled oligonucleotide corresponding to the respective receptor (Table 2). Lanes 1-15 contain RNA samples representative of the 50 cancerous breast tissue samples obtained at biopsy. These were examined for the growth factor receptors and β-Actin as indicated. Control samples, obtained when reverse transcriptase was ommitted from the initial cDNA synthesis of each sample, gave no signal. In some samples (CNTFR, ER and IL-6R) a smaller hybridising PCR product was identified. These bands were attributed to alternative splicing (Koehorst et al., 1993; Horiuchi et al., 1994).

### EXAMPLE 1

### Breast Cell Lines

Cell lines 184 (Stampfer and Bartley, 1985) and 184B5 (Walen and Stampfer, 1989) were derived from non malignant breast epithelial cells; BT-483 (Lasfargues et al., 1978), MCF-7M (Soule et al., 1973), MDA-MB-134 (Cailleau et al., 1974), MDA-MB-361 (Cailleau et al., 1978) and T-47D (Keydar et al., 1979) cell lines originated from estrogen receptor (ER) positive breast cancer cells; BT-20 (Lasfargues and Ozzello, 1958), BT-549 (Lasfargues et al., 1978), MDA-MB-231 (Cailleau et al., 1974), SK-BR-3 (Trempe and Fogh, 1973) cell lines originated from ER negative breast cancer cells. The HBL-100 cell line is an ER negative transformed cell line, originating from normal lactating breast (Caron de Fromentel et al., 1985).

### EXAMPLE 2

### Tissue Culture

Breast cell lines were grown in monolayer in RPMI-1640 medium containing 10% (v/v) bovine calf serum ((v/v) BCS) at 37” C in a fully humidified atmosphere, containing 10% (v/v) CO₂ in air. Cell lines were passaged by treatment with 0.05% (w/v) trypsin and 0.02% (w/v) EDTA.

### EXAMPLE 3

### Reverse Transcriptase Polymerase Chain Reaction

Total RNA was extracted from the cell lines and primary breast cancer tissue as previously described (Buckley et al., 1993).

First strand cDNA synthesis was performed on 1 82 g of total RNA. Reverse transcription was carried out at 42° C. for 60 min in 20 μl of 50 mM Tris.HCl pH 8.3, 20 mM KCl, 10 mM MgCl₂, 5 mM dithiothreitol, 1 mM of each dNTP, 20 μg/ml oligo(dT) and 12.5 units of AMV reverse transcriptase (Boehringer Mannheim). Control reactions were performed for each RNA sample under identical conditions except that reverse transcriptase was omitted from the reaction. The reverse transcription reaction mixture was diluted to 100 μl with water and 5 μl was used for each PCR reaction.

PCR reactions were carried out in 50 μl of reaction buffer (Boehringer Mannheim) containing 200 μM of each dNTP, 1 μM of each primer and 2.5 units of Taq polymerase (Boehringer Mannheim). The oligonucleotides used for amplification of cDNA are shown in Table 2. After an initial denaturation of 2 min at 96° C. PCR was performed for 30 cycles in a Hybaid Omnigene Thermal Cycler (Integrated Sciences). Each cycle consisted of 30 sec denaturation at 96° C., 30 sec annealing at 60° C. and 2 min polymerisation at 72° C. 20 μl of the reaction mixture was electrophoresed on a 1% (w/v) agarose gel and DNA transferred to a nylon membrane (hybond-N+, Amersham). Southern blots were performed as described previously (Reed and Mann, 1987). Hybridisation was carried out with end-labelled oligonucleotides internal to the respective cDNA sequences (Table 2).

### EXAMPLE 4

### Binding Studies

Receptor binding assays were performed using radioiodinated LIF (¹²⁵I-LIF) and OSM (¹²⁵I-OSM). The radioiodination of LIF and OSM and binding assays were essentially performed as previously described (Hilton et al., 1991; Hilton and Nicola, 1992). Briefly, 50 μl aliquots containing 1×10⁷ cells, suspended in RPMI-1640 medium containing 10% (v/v) BCS, were placed in Falcon tubes with 40 μl of the respective radioiodinated ligand at 1×10⁵ cpm per 40 μl, with or without greater than a 40-fold excess of unlabelled ligand. Incubation was carried out at room temperature for 60 min and cells were resuspended and layered over 180 μl of (v/v) BCS. Cell associated and free radioiodinated ligand were separated by centrifugation. The pellet and supernatant were subsequently counted in a γ-counter. Specific binding was estimated by subtraction of non specific binding from binding with ¹²⁵I-ligand (total binding). Number of cell surface receptors and dissociation constant were calculated by Scatchard analysis.

### EXAMPLE 5

### Biological Assays

Proliferation of the cell lines was measured in monolayer culture in 24 well Costar cluster plates. Cells were plated at an initial density of 10 000 cells/ml and cultured in 500 μl RPMI-1640 supplemented with 10% (v/v) (v/v) BCS and with each growth factor as indicated (LIF, 1000 U/ml; IL-6, 100 ng/ml; OSM, 10 ng/ml; CNTF, 10 ng/ml; IL-11, 100 ng/ml). These concentrations are maximally active in other systems (Nandurkar et al., 1996; Hilton et al., 1994; Zhang et al., 1994; Tanigawa et al., 1995). After 7 or 10 days at 37° C. in a fully humidified atmosphere containing 10% (v/v) CO₂ in air, cells were trypsinised and counted using a haemocytometer and an inverted microscope. Cell viability was assessed using eosin exclusion. Results were expressed as a percentage of the corresponding untreated control value for that experiment.

### EXAMPLE 6

### Clonogenic Assays

Clonogenic potential of cells following monolayer culture was assessed in a semi-solid culture medium. Cells were cultured in triplicate in 35 mm Petri dishes containing 1 ml Iscove's modified Dulbecco's medium (IMDM) supplemented with 25% (v/v) (v/v) BCS, 0.3% (w/v) agar with final concentration of growth factor as outlined above, and with 200 cells per ml for control cultures Cultures were maintained at 37° C. in a humidified incubator with 5% (v/v) CO₂ in air. After 14 days, colonies were enumerated using a dissecting microscope. A colony was defined as a clone of greater than 40 cells. All cultures were performed in triplicate.

### EXAMPLE 7

### Cytokines

Human LIF was produced using the pGEX system, essentially as described (Gearing et al., 1989), human IL-6 was from Ludwig Institute for Cancer Research, (Melbourne, Australia), human CNTF was purchased from AMRAD Operations Ltd. (Melbourne, Australia) and human OSM and IL-11 were purchased from Pepro Tech (Rocky Hill, N.J., USA).

### EXAMPLE 8

### Statistical Analysis

Statistical analysis of data was performed using the paired and unpaired Student's T-test.

### EXAMPLE 9

### Growth of Primary Breast Tissue

Sterile normal breast tissue was obtained from reduction mammoplasty surgery. Fat was dissected away and the remaining ductal tissue minced finely, suspended in ‘dissociation media’ (DME/Hams F12 containing 10 ng/ml EGF, 1 μg/ml insulin, 0.5 μg/ml hydrocortisone, 10 ng/ml cholera toxin, 300 U/ml collagenase, 100 U/ml hyaluronidase and 1 mg/ml BSA) and agitated overnight at 37° C. After 18 hours the mixture was centrifuged at 600 RCF/5 min, the supernatant discarded and the remaining cell pellet washed twice in RPMI-1640 supplemented with 10% (v/v) (v/v) BCS. An alliquot of the cells was then placed in an 80 cm² tissue culture flask (Nunc) in ‘breast media’ (DME/Hams F12 containing 10 ng/ml EGF, 1 μg/ml insulin, 0.5 g/ml hydrocortisone, 10 ng/ml cholera toxin, 1 mg/ml BSA supplemented with 10% (v/v) (v/v) BCS) for 24 hour at 37° C. in a fully humidified atmosphere, containing 10% (v/v) CO, in air. Single cells adhered to the culture flasks and from these islands of cells epithelial cells grew. The media was subsequently removed and replaced with serum-free breast media. Partial trypsinisation removed any contaminating fibroblasts and the cells remaining were 95-100% epithelial. Cells were subsequently grown for approximately 30 days in the serum free breast media.

### EXAMPLE 10

### Cell Cycle Analysis

Cell cycle analysis was performed in serum-free medium (Sigma). Analysis was performed 2-4 days after cells were washed and re-cultured in serum-free medium. Growth factors were added to the medium as indicated. At the times shown thereafter, cells were harvested with 0.05% (w/v) trpsin-0.02% (w/v) EDTA. The cells were resuspended in serum-free tissue culture medium and after cell counting using a hemacytometer, stained for later DNA analysis by the addition of 0.25% prothidium iodide in the presence of 0.2% (v/v) Triton X-100. DNA histograms were obtained by using a FACScan flow cytometer (Becton Dickinson Immunocytochemistry Systems) and the cell cycle phase distribution was estimated by using the manufacturer's DNA analysis software (Cellfit). Each histogram contained 10,000 events.

### EXAMPLE 11

### RNA Isolation and Northern Analysis

Cells havested from duplicate flasks were pooled and poly A+ mRNA extracted by an oligo-dT cellulose procedure (Boehringer Mannheim). Northern analysis was performed using 5 μg RNA per lane. Membranes (Hybond-C extra-Amersham) were hybridised (42° C. overnight) with probes labelled with α-³²P dCTP (bresatec). The membranes were washed at a stringency of 0.2× SSC (30 mM NaCl, 3 mM sodium citrate, pH 7.0)-0.1% (w/v) sodium dodecyl sulfate at 65° C. and exposed to Kodak X-Omat film at −70° C. mRNA loading was estimated by hybridising membranes with a 1.3 kb cDNA complementary to GAPDH.

### EXAMPLE 12

### Analysing Growth Factor/Receptor Expression by RT-PCR

Initial experiments have examined expression of several receptors, including gp 130, LIF, G-CSF, GM-CSF, CNTF, IL-2, 3, 6, 7 & 11 and their associated ligands (FIG. 1). Preliminary results have indicated the expression of both ligand and receptor in the case of IL-6, LIF and CNTF. The expression of IL-11 receptor was observed in most of the breast samples. The signalling molecule gp130 was also expressed (as expected). The expression of such receptors as G-CSF, GM-CSF and IL-2 appeared to be less consistent in the breast samples.

### EXAMPLE 13

### Function of Growth Factors/Receptors on Breast Cell Growth

The function of the growth factors and receptors identified in initial mRNA and protein studies of breast cancer cells have also been investigated. Breast cell lines were initially examined for changes in cell proliferation in suspension cultures containing the growth factors of interest. Preliminary experiments have examined the growth of several of the breast cancer cell lines in cultures with maximal concentrations of LIF, IL-6 and OSM. These cultures have indicated that two of these growth factors have inhibitory effects on cell proliferation. FIGS. 2A, 2B and 2C show the proliferation of MCF-7M cells following 1 week in suspension culture. This proliferation data indicates that OSM and LIF may inhibit cell growth. FIG. 3 shows the viability of the MCF-7M cells following a clonogenic assay. Results indicate that the effects of the two growth factors are enhanced after this assay.

This inhibition of cell proliferation by OSM has been demonstrated in cell lines MCF-7M and BT-549. LIF induced inhibition in cell line MCF-7M. It is interesting to note that IL-6 did not appear to be able to induce inhibition when compared to the control (FIG. 2A).

FIG. 4 depicts four photomicrographs of MCF-7M breast cancer cells treated with maximal concentrations of IL-6, OSM and LIF for 1 week in liquid culture. Cells grown in the presence of IL-6 appeared morphologically similar to the control; several of the cells grown in LIF appeared larger than the control cells; the cells grown in the presence of OSM showed more abundant cytoplasm and vacuolation. This morphological change in the OSM treated cells was consistent with features of cell differentiation. These findings suggested that ligand-induced growth inhibition in breast cancer cell lines may be associated with an apparent induction of differentiation.

FIGS. 5A and 5B show cells from the BT-549 cell line that have been grown in liquid culture in the presence of IL-11. As well as the growth inhibition seen previously on other cell lines with OSM, the BT-549 cell proliferation was inhibited by IL-11.

### EXAMPLE 14

### Examination of Breast Tissues

The inventors developed culture techniques that allow the growth of normal breast cells in vitro. A total of 4/4 normal breast samples have been successfully cultured and continued to proliferate for several weeks.

RT-PCR analysis from three of these primary normal breast samples have indicated that gp 130, LIFR, LIF and OSM are expressed in these cells. FIG. 6 shows the growth of one of these primary samples in suspension culture with the various growth factors. These data show that OSM profoundly inhibited the proliferation of these normal cells.

### EXAMPLE 15

### Inhibition of Proliferation of MCF-7 Cells by Oncostatin M (OSM)

The results are shown in FIG. 7. 10⁴ MCF-7 cells were cultured in 500 μl RPMI/10% (v/v) BCS with the indicated concentrations of OSM. At 2, 4 and 6 days, viable cells were counted using a hemacytometer. Results are from 3 experiments, each performed in triplicate. These data demonstrate that while control cells grow in a exponential fashion over the 6 day time period, there was inhibition of cellular proliferation as a result of treatment with OSM.

### EXAMPLE 16

### MCF-7 Cells are Inhibited in a Dose-dependent Fashion by Oncostatin M (OSM)

The results are shown in FIG. 8. 10⁴ MCF-7 cells were cultured in 500 μl RPMI/10% (v/v) BCS with the indicated concentrations of OSM. After 7 days viable cells were counted, and cell number expressed as a percentage of the corresponding untreated control value. Results are from experiments each performed in triplicate. Results indicate that treatment of MCF-7 cells with pg/ml quantities of OSM results in decreases in cellular proliferation while a concentration of 10 ng/ml OSM results in optimal inhibition.

### EXAMPLE 17

### Effect of OSM on Cell Cycle

The results are shown in FIG. 9. MCF-7 cells treated with OSM while growing in serum-free medium were harvested by treatment with trypsin and stained for DNA content analysis by flow cytometry. Cell cycle distributions were calculated by computer fitting of the resultant histograms. FIG. 9A represents a typical experiment indicating that the percentage of cells in S phase following treatment with OSM decreases from approximately 15% to 8% over a 72 hour time period. FIG. 9B represents data from 2 experiments (performed in triplicate) where the number of cells in S phase are represented as a percentage of the corresponding untreated control value. At the initial time point of 12 hour there is a marked decrease in the S phase cells in OSM treated cells. By 24 hour the number of cells in S phase was 50% of control cells. There was concomitant increase in the percentage of cells in G₁ phase, demonstrating that OSM is inhibiting a rate limiting step in progression through G₁. Similar results were seen when cells were grown in RPMI containing 10% (v/v) BCS.

### EXAMPLE 18

### Effect of EGF and OSM on Cell Cycle

The results are shown in FIG. 10. MCF-7 cells treated with OSM, Epidermal Growth Factor (EGF) or both OSM and EGF while growing in serum-free medium were harvested by treatment with trypsin and stained for DNA content analysis by flow cytometry. Cell cycle distributions were calculated by computer fitting of the resultant histograms. Results represent the combined data from 3 experiments performed in triplicate) where the number of cells in S phase are represented as a percentage of the corresponding untreated control value. The S phase fraction is decreased in cells treated with OSM, and this is maintained over a 6 day time period. Cells treated with EGF (thought to be a mitogenic stimuli in some breast cancers) and OSM also demonstrate a 50% decrease in S phase fraction.

### EXAMPLE 19

### Cell Morphology After Exposure to OSM

The results are shown in FIG. 11. MCF-7 cells from control cultures and appearance of cells after 7 days in OSM. A) Control cells, 10× magnification. B) OSM treated cells, 10× magnification. C) OSM treated cells, 40× magnification. D) OSM treated cells, 100× magnification. Striking changes in the morphology of cells treated with OSM are apparent. MCF-7 cells exposed to OSM appeared to draw apart from neighbouring cells, and to develop a more fibroblastic phenotype. This was associated with the appearance of decreased intercellular adhesion and the development of pseudopodia-like processes.

### EXAMPLE 20

### Effect of OSM on the Expression of Transforming Growth Factor α (TGFατ, Epidemal Growth Factor Receptor (EGFR), Prolactin Receptor (PRLR), Estrogen Receptorm (ER) and LIF mRNA

The results are shown in FIG. 12. Cells growing in the presence of 10% (v/v) BCS were treated with OSM (10 ng/ml) and at the indicated time points duplicate 150 cm² flasks were harvested and mRNA extracted for Northern analysis. Results for control cells (C) are also shown. The same filter has been probed successively with a ³²P-labelled cDNA corresponding to each mRNA species. mRNA loading was evaluated by reprobing the filter with a fragment complementary to GAPDH. mRNA species of the following sizes were obtained: TGFα, 4.8 kb; PRLR, 10.5 and 8.6 kb; EGFR, 10.5 and 5.8 kb; ER, 6.5 and 3.8 kb and LIF, approx. 4.8 kb.

Northern analysis demonstrates that as a result of cells being exposed to OSM the abundance of EGFR mRNA is elevated at least 5-fold between 4-12 hours. The EGFR transcript decreases to control levels by 24 hour. The abundance of Transforming Growth Factor α (TGFα) transcript in OSM treated cells appears to be equivalent to control cells over this time period. However, OSM appears to down regulate the level of expression of both Estrogen Receptor (ER) and Prolactin Receptor (PRLR) mRNA. After only 2 hours exposure to OSM the levels of these two receptors is down regulated and this is maintained for 48 hours. OSM also upregulates the level of LIF transcript in MCF-7 cells over the 14 hour time period.

### EXAMPLE 21

### ER Expression in OSM and EGF Treated Cells

The results are shown in FIG. 13. MCF-7 cells were grown on chamber slides for 6 days in RPMI/10% (v/v) BCS with the following growth factors, prior to being stained with an antibody specific for ER. A) Control, B) OSM, C) EGF, D) OSM and EGF. Cells stained brown indicate ER positivity. Results indicate that 90% of control cells have stained positive for ER and cells treated with EGF have approximately 60% ER positivity. Cells treated with OSM show 50% of cells staining very weakly for ER, indicating a down regulation of ER protein levels after treatment with OSM. Furthermore, this down regulation of ER protein is more dramatic when cells are treated with both OSM and EGF as only approximately 10% of cells have stained positively for ER. Morphologically it can be seen that cells treated with OSM appear larger and more vacuolated than control cells. The morphological changes are more striking when cells are treated with OSM and EGF: cells are larger than control cells and have more abundant cytoplasm and cytoplasmic processes.

### EXAMPLE 22

### RT-PCR Analysis of Receptor Expression on Breast Cancer Cell Lines

Initial experiments examined the expression of various receptors on 12 breast cell lines. These receptors included the LIF receptor (LIFR), IL-6R, IL-11R, CNTFR, the common gp130 signalling molecule and receptors for IL-2, 3, 6, 7 & 11, G-CSF, GM-CSF, growth hormone (GM) and prolactin (PRL) (FIG. 14). Both GHR and PRLR were expressed primarily in estrogen receptor (ER) positive cell lines with inconsistent expression in ER negative cells. GHR was also expressed in the breast cell lines derived from normal tissue, whereas PRLR was not expressed in these cells.

All cell lines expressed the signalling molecule gp130, and expression of the specific receptor components for IL-6, LIF, IL-11 and CNTF was observed in the majority of cell lines studied. Expression of the LIFR was ubiquitous and appeared equivalent to the level of gp130 as assessed by RT-PCR IL-11R expression was also observed in all of the cell lines except SK-BR-3 (a line originating from an ER negative breast carcinoma). However, expression of the IL-6R in the cell lines appeared variable, consistent with the variable biological effects reported for IL-6. For example expression of the IL-6R in MCF-7, T-47D and SKBR3 cell lines is consistent with previous reports of variable effects of IL-6 in these cell lines. Equally the lack of IL-6R expression in the MDA-MB-231 cell line correlates well with reports describing its lack of activity on these cells (Danforth and Sgagsis, 1993).

The CNTFR was readily detected in cell lines that expressed the ER, but with no expression observed in cell lines derived from normal breast nor ER negative cell lines. The pattern of expression was thus similar to PRLR expression.

In contrast to the widespread expression of gp130 and associated receptors, expression of receptors for G-CSF, GM-CSF, IL-2 and IL-3 was highly variable in these breast cell lines. G-CSFR was expressed only in the BT-483 cell line. While the β common signalling subunit shared by GM-CSF, L-3 and IL-5 was detected in 6 cell lines the specific GM-CSFRα and IL-3Rα chains were expressed in only 2 cell lines. The IL-2Rγ common signalling subunit, shared by IL-2, 4, 7, 9 and 13, was expressed in 2 of the breast cell lines, while for example, the IL-7Rα chain was not expressed in any cell lines.

Because of the widespread expression pattern of the gp130 molecule and associated receptors in the majority of breast cell lines compared with the variable expression of other cytokine receptors, we elected to focus on the gp130 sub-family in this study.

### EXAMPLE 23

### Action of Cytokines on Growth of Breast Cell Lines

The action of IL-6, LIF, OSM CNTF and IL-11 was examined on 4 breast cancer cell lines grown in monolayer culture. Striking changes in the morphology of cells were observed. FIG. 15 compares morphology of untreated cells with cells exposed to OSM. MCF-7 cells exposed to OSM appeared to draw apart from neighbouring cells, and to develop a more fibroblastic phenotype. This was associated with the appearance of decreased intercellular adhesion or cellular contraction. These changes were quite marked by day 14. Transformation to a fibroblastic phenotype was also observed in the BT-549 and MDA-MB-231 cell lines exposed to OSM, with elongation of cells and loss of intercellular contact. In contrast T-47D cells cultured with OSM, became more rounded in appearance.

Experiments were performed to determine whether these morphological changes were associated with alterations in cell growth. The proliferation of four breast cancer cell lines was examined in monolayer cultures containing either IL-6, LIF, OSM, CNTF or IL-11. In this assay, significant inhibition of cellular proliferation by OSM in ¾ cell lines, IL-11 in 2/4 cell lines and by IL-6 and LIF in ¼ cell lines was observed.

The MCF-7 cell line exhibited a biological response following treatment with this family of growth factors. Results of 9 experiments examining action of IL-6, LIF and OSM, and 5 experiments examining action of CNTF and IL-11 on the MCF-7 cell line are presented in FIG. 16. In control cultures of MCF-7 cells the absolute cell number increased from 10⁴/ml to 5×10⁻-1.1×10⁵/ml during the 1 week culture period. The most dramatic effect on cell proliferation was seen after 7 days exposure to OSM, with up to 94% inhibition and a mean of 85% inhibition in 9 experiments (p<0.001). This action of OSM was maximal at concentrations of greater than 10 ng/ml. Exposure to LIF for 7 days resulted in an average of 37% growth inhibition (p<0.01). The effect of IL-11 and IL-6 was less marked. The mean inhibition observed in response to IL-6 was 23% (p<0.01). In five experiments using IL-11a mean of 27% inhibition (p=0.02) was observed. In contrast, there was no effect on cellular proliferation in five experiments in which cells were treated with CNTF.

To further address the inhibitory effect of these molecules on MCF-7 cells we also examined the clonogenic potential of cells following growth in monolayer cultures for 7 days. The frequency of clonogenic MCF-7 cells in control cultures was 20-60% (n=9 experiments). With OSM, LIF and IL-6 the growth inhibitory effect observed in monolayer culture were also detected in agar cultures. Results of 9 experiments (FIG. 17) showed significant reduction of colony formation by cells which had been exposed to IL-6 (p<0.01), LIF (p<0.01) and OSM (p<0.01). In 5 experiments exposure to IL-11 in monolayer culture did not have detectable effect on subsequent clonogenicity (p=0.65). As in the monolayer cultures, exposure to CNTF had no effect on clonogenic potential of these cells.

Results of treatment of the cell line BT-549 with growth factor for 10 days are presented in FIG. 18 (n=6 experiments). The number of BT-549 cells in control cultures increased from 10⁴/ml to 4-9×10⁴/ml during the 10 day culture period. Mean growth inhibition of 60% followed treatment with OSM, with up to 80% inhibition observed (p<0.01). This inhibition was also apparent at 7 days (mean 60%, p<0.01). Similarly, inhibition of up to 63% (mean 56%, p<0.01) in response to IL-11 was observed at 10 days however after 7 days in culture this effect was less evident (mean 30%, p=0.17). As expected from the results shown in FIG. 14 with no detectable mRNA for IL-6R and very low levels of CNTFR mRNA.

FIG. 19 depicts 8 experiments using the ER negative cell line, MDA-MB-231. Inhibition of proliferation of up to 65% (mean 54%, p<0.01) was observed in cells exposed to OSM. Treatment with LIF did not result in significant effects on cellular proliferation. These cells did not express CNTFR (FIG. 14) and did not respond to CNTF. Similarly the level of IL-6R expression was barely detectable (FIG. 14).

### EXAMPLE 24

### Analysis of Receptor Protein Expression

The response of BT-549 and MDA-MB-231 cells to OSM but not to LIF was unexpected given that both growth factors utilize the LIF-gp130 heterodimer. Experiments were, therefore, performed to document LIFR expression and binding of LIF to the surface of these cells. Binding assays were performed to monitor incorporation of the respective radio-labelled ligand. Four breast cancer cell lines (T-47D, MDA-MB-231, MCF-7 and HBL-100) were examined with ¹²⁵I-LIF and ¹²⁵I-OSM. Table 3 shows the specific binding of OSM and LIF. All four of the cell lines demonstrated specific binding Three of the cell lines showed increased binding of OSM compared with LIF. Binding of radiolabelled LIF was comparable for all cell lines, In contrast there was approximately 10-fold reduced binding of radiolabelled OSM to HBL-100 cells compared with the other cell lines (a cell line derived from normal lactating breast). Thus the failure of BT-549 cells and MDA-MB-231 cells to respond to LIF could not be attributed to lack of expression of LIFR mRNA (FIG. 14) nor to lack of receptor protein expression (Table 3).

Binding of ¹²⁵I-LIF to the MCF-7 cell line (FIG. 20A) revealed a single class of high affinity binding sites for LIF with an estimated 57 receptors per cell and a dissociation constant of 14.6 pM. In contrast, and in keeping with the results presented in Table 3, MCF-7 cells showed an estimated 990 receptors per cell and a dissociation constant of 2 nM for ¹²⁵I-OSM. Results obtained with the HBL-100 cell line also demonstrated high affinity binding of ¹²⁵I-LIF. HBL-100 cells showed an estimated 27 receptors per cell and a dissociation contstant of 7.49 pM. The number of LIF binding sites observed on these cells is comparable with estimates of receptor number for other tissues (Hilton et al., 1991). FIG. 21B shows a Scatchard analysis depicting binding of ¹²⁵I-OSM to the MDA-MB-231 cell line. MDA-MB-231 cells also showed a single class of high affinity binding sites for OSM, with an estimated 124 receptors per cell and a dissociation constant of 92 pM.

### EXAMPLE 25

### Receptor Expression by RT-PCR on Primary Breast Cancer Samples

Based on the aforementioned results, the inventors sought to determine whether the gp130 sub-family of receptors might also be expressed on fresh tumor samples. Although it was possible that these receptors might be expressed on normal breast cells contaminating these tissue samples, the concordance between results from primary samples and analysis of cell lines suggested that this was not the case.

Typical results for expression of the gp130 sub-family of receptors from 50 clinical samples of malignant breast tissue are shown in FIG. 21. This analysis showed a strikingly similar pattern of expression of gp130 associated receptors to that observed on breast cancer cell lines. Expression of gp130, LIF and IL-11 receptors was detected on 96%, 96% and 98% of the samples respectively. By comparison, IL-6 receptor was detected in only 80% of the samples. This was consistent with the variable expression pattern of this receptor relative to LIF and IL-11 receptors that was observed on cell lines. CNTFR expression was observed in 94% of the primary breast cancer samples. This was more frequent than CNTFR expression in the cell lines, and the correlation with ER (only 68% of samples were ER positive) was less marked. It was interesting, however, that the three samples that were CNTFR negative were also ER negative. Thus the widespread expression of members of the gp130 sub-family of receptors was observed not only in breast cancer cell lines but in the majority of samples obtained from malignant breast tissue.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2
Sequence of Oligonudeotides

| | size cDNA | 5′ sequence/3′ sequence | internal sequence | |
|---|---|---|---|---|
| Gene | (bp) | (5′-3′) | (5′-3′) | Reference |

| | | | | |
|---|---|---|---|---|
| gp 130 | 811 | GAGGTGTGAGTGGGATGGTGG | GGGCAACACACAAGTTTGCTGATTG2 | Hibi et al, 1991 |
| | | GCTGCATCTGATTTGCCAAC¹ | | |
| IL-6R | 748 | GTTTCAGAACAGTCCGGCCG | CAGGAGCCGTGCCAGTATTCCCAGC⁴ | Yamasaki et at., 1988 |
| | | CTTGCCTTCGTTCAGAGCCC³ | | |
| LIFR | 660 | CCCTCTGGAACAGGCCGTGG | GAAGTTTGCATTGAAAACAGGTCCCG⁶ | Gearing et at., 1991 |
| | | CAAGGGGCAGTTTGTATGGCC⁵ | | |
| IL-11R | 509 | CTGAGTTCTGGAGCCAGTAC | GTGACTGAGGTGAACCCACTGGCTG⁸ | Nandurkar et al., 1996 |
| | | GGTGTGGTTGGAGGGAGGGC⁷ | | |
| CNTFR | 649 | GTGGGCCTGCTGTGCTGTGC | CGCCGCAGTTGTCTACGCCCAGAG¹⁰ | Davis et al., 1991 |
| | | CCAGCCGGCGAGGGTTGCTG⁹ | | |
| GCSFR | 1034 | GCTGCATCTAAAGCACATTG | GACCTGGGCACAGCTGGAGTGGGTG¹² | Fukunaga et al., 1990 |
| | | GAGATGGTGAGAGCCTGGGCTG¹¹ | | |
| PLR | 670 | CAGACTACATAACCGGTGGC | CAAGCAGTACACCTCCATGTGGAGG¹⁴ | Boutin et al., 1989 |
| | | TGGCATCCCAAGGCACTCAG¹³ | | |
| GHR | 702 | CAGATCCACCCATTGCCCTC | GGCGAGTTCAGTGAGGTGCTCTATG¹⁶ | Leung et al., 1987 |
| | | CGCCATCCTTCACCCCTAGG¹⁵ | | |
| GM-CSFR β | 710 | CCACCAGGTACTGGGCCAGG | GCACCGGCTACAACGGCATCTGGAG¹⁸ | Hayashida et al., 1990 |
| | GAGGGACCAGTTGCACCTGC¹⁷ | | | |
| GM-CSFR α | 928 | GGAAGGGAGGGTACCGCTGC | CTGTACCTGGGCGAGGGGTCCGACG²⁰ | Crosier et al., 1991 |
| | CTTGACCACCACCCTGCCTC¹⁹ | | | |
| IL-2R γ | 776 | CCCCTCCCAGAGGTTCAGTG | CAGCAGCTCTGAGCCCCAGCCTACC²² | Takeshita et al., 1992 |
| | | AGACACACCACTCCAGGCCG²¹ | | |
| IL-3R α | 888 | GCCGACTATTCTATGCCGGC | CCGTCCGAGTGGCCAACCCACCATT²⁴ | Kiamnura et al., 1991 |
| | | CGTTTTGGAAGCTGTCACCG²³ | | |
| ER | 711 | GTGTACAACTACCCCGAGGG | CGCCAACGCGCAGGTCTACGGTCAG²⁶ | Green et al., 1986 |
| | | CTCATGTCTCCAGCAGACCC²⁵ | | |
| β-ACTIN | 721 | CTTCCCCTCCATCGTGGGGC | CGACGAGGCCCAGAGCAAGAGAGGC²⁸ | Ponte et al., 1984 |
| | | GTTTCGTGGATGCCACAGGAC²⁷ | | |

1-28 Corresponds to SEQ ID Nos. 1-28

TABLE 3
BINDING OF hOSM AND hLIF TO HUMAN BREAST CANCER CELL
LINES

specific binding(cpm)/10⁶ cells

| Cell line | OSM | LIF |
|---|---|---|
| T47D | 1400 +/− 20 | 380 +/− 10 |
| MIDA-MB-231 | 2980 +/− 460 | 450 +/− 120 |
| MCF-7M | 1250 +/− 90 | 350 +/− 100 |
| HBL-100 | 140 +/− 10 | 590 +/− 30 |

### BIBLIOGRAPHY

Antman, K. H. 1992. Dose intensive therapy in breast cancer. In Armitage, J O and Antman, K H, eds., High-dose cancer therapy: Pharmacology, Hematopoietins, stem cells. Williams & Williams, Baltimore; 701-718.

Ardizzoni A. et al. 1994. Br J Cancer 69: 385-391.

Balawadjer I., Antich P. P., Bland J. 1983. Cancer 51: 574.

Basser R. L. et al 1995. Clin Can Res. 1:715-721.

Bonadonna G. and Valagussa P. 1981. N Eng J Med 304: 10-15.

Bronchud M. H. et al. 1989. Br 3 Cancer 60: 121-125.

Buckley, M. F., Sweeney, K. J. E., Hamilton, J. A., Sini, R L., Manning, D. L., Nicholson, R I., deFazio, A, Watts, C. K. W., Musgrove, E. A. and Sutherland, R. L. (1993) Oncogene, 8: 2127-2133.

Cailleau R, Olive, M. and Cruciger, J. (1978) In vitro, 14: 911-915.

Cailleau R, Young, R., Olive, M. and Reeves, W. J. (1974) J. Natl. Cancer. Inst., 53: 661-674.

Cailleau R. et al. 1974. J. Natl. Cancer. Inst., 53: 661-674.

Cailleau R. et al. 1978. In vitro 14: 911-915.

Carmo-Pereira J. et al. 1987. Br J Cancer 56: 471-473.

Caron de Fromental, C., Nardeux, P. C., Soussi, T., Lavialle, C., Estrade, S., Carloni, G., Chandrasekaran, K. and Cassingera, R. (1985) Exptl. Cell. Res., 160: 83-94.

Chen, L. et al. 1991. J Biol Regulators and Homeostatic agents. 5(4):125.

Chrysogelos, and Dickson, R. 1994. Breast Cancer Res Treat 29: 29.

Davis S. et al. 1991. Science 253: 59-63.

Danforth, D. N. and Sgagias, M. K. (1993) Cancer Res., 53: 1538-1545.

Dedhar, S. et al. 1988. PNAS 9253.

Dickson, R. B. and Lipmann M. E. 1992. Semin Oncol. 19:286.

Early Breast Trial Collaborative Group. Lancet 1992; 339(1):71.

Eddy D. M. 1992. J Clin Oncol 10: 657-670.

Ethier, S. P. et al. 1993. Cancer Res 53: 627.

Ferguson J. E. et al. 1993. Br J Cancer 67: 825-829.

Fisher B., Slack N. H., Bross I. D. H. 1969. Cancer 24: 1071.

Focan C. et al. 1993. J Clin Oncol 11: 1253-1263.

Fukunaga R. et al. 1990. PNAS USA 87: 8702-8706.

Gearing, D. P., King, J. A., Gough, N. M. and Nicola, N. A. (1989) EMBO J, 8: 3667-3676.

Gearing et al. 1991. EMBO J 10: 2839-2848.

Gearing, D. P. et al. 1992. Sci 255: 1434.

Gianni. M: et al. 1992. Pro Am Soc Clin Oncol 11: 60.

Green, A. R. et al. 1991. EMBO J 10: 4153.

Haagensen, C. D. 1986. Diseases of the breast (ed 3). Philadelphia, WB Saunders, p 635.

Halstead W. S. 1907. Ann Surg 46: 1.

Harris, A. L. 1994. Breast Cancer Res Treat 29: 1.

Hibi M. et al. 1991. Cell 63: 1149-1157.

Hilton, D. J., et al. 1991. J Cell Phys 146: 207.

Hilton D. J. and Nicola N. A. 1992. J Biol Chem 267: 10238-10247.

Hilton, D. J., Hilton, A. A., Raicevic, A., Rakar, S., Harrison-Smith, M., Gough, N. M., Begley, C. G., Metcalf, D., Nicola, N. A. and Willson, T. A. (1994) EMBO J, 13(20): 4765-4775.

Hilton, D. J. and Nicola, N. A. (1992) J. Biol. Chem., 267: 10238-10247.

Hilton, D. J., Nicola, N. A. and Metcalf, D. (1991) J. Cell Phys., 146: 207-215.

Horiuchi, S., Koyanagi, Y., Zhou, Y., Miyamoto, H., Tanaka, Y., Waki, M., Matsumoto, A., Yamamoto, M. and Yamamoto, N. (1994) Eur. J. Immunol., 24: 1945-1948.

Hoekman K. et al. 1991. J Natl Cancer Inst 83: 1546-1553.

Hrynuik W. and Bush H. 1984. J Clin Oncol 2: 1281-1288.

Hrynuik W. and Levine M. N. 1986. J Clin Oncol 4: 1162.

Jain K. K., Casper E. S. et al. 1985. J Clin Oncol 3: 818-826.

Jones R. B. et al. 1987. J Clin Oncol 5(2): 172-177.

Kacinski B. M. et al. 1991. Oncogene 6: 941-952.

Keydar I., Chen, L., Karby, S., Weiss, F. R, Delarea, J., Radu, M., Chaitcik, S. and Brenner, H. J. (1979) Eur. J. Cancer, 15: 659-670.

Koehorst, S. G. A., Jacobs, H. M., Thijssen, J. H. and Blankenstein, M. A. (1993) J. Steroid Biochem. Mol. Biol., 43: 227-233.

Komminoth P and Long AA. 1993. Virchows, Archiv B Cell Pathol. 64:67.

Lalisang, R. et al. 1994. Pro Am Soc Clin Oncol 13: 60.

Lasfargues E. Y. and Ozello L. 1958. J Natl Cancer Inst 21: 1131-1147.

Lasfargues E. Y. et al. 1978. J Natl Cancer Inst 61: 967-978.

Li, S. et al. 1992. J Cell Physiol. 153: 103.

Malik N. et al. 1989. Mol Cell Biol 9: 2847-2853.

Marchner N. et al. 1994. Semin Oncol 21(Suppl 1): 10-16.

Moreau J. F. et al. 1988. Nature 336: 690-692.

Musgrove, E. A. et al. 1994. PNAS 91:,8022.

Nandurkar, H. H., Hilton, D. J, Nathan, P., Willson, T., Nicola, N. and Begley, C. G (1996) Oncogene, 12: 585-593.

Negata S. et al. 1986. Nature 319: 415-418.

Negro A. et al. 1991. Eur J Biochem 201: 289-294.

Nemoto, T., Vana J., Bedwani R. N., et al. 1980. Cancer 45: 2917.

Neri B. et al. 1993. Cancer Invest 11: 106-112.

Nuovo G. J et al. 1991. Am J Pathol 139(4): 847-854.

Osborne, L. K. 1991. In Harris J. R. et al. (eds). Breast Diseases, ed 2. Philadelphia, J.B. Lippincott, p.301.

Paul S. R. et al. 1990. PNAS USA 87: 7512-7516.

Peters W. P. et al. 1993. J Clin Oncol 11: 1132-1143.

Petersen, O. W. et al. 1992. PNAS 89: 9064.

Piccart M. J. et al. 1995. Ann Oncol 6: in press.

Ponte P. et al. 1984. Nucl Acids Res 12:1687-1696.

Reed K. C. and Mann D. A. 1987. Nucl Acids Res 13: 7207-7221.

Rozencweig M. et al. 1984. J Clin Oncol 2: 275-286.

Saiki, R. K. et al. 1985. Sci 223: 487.

Scinto A. F. et al. 1995. Ann. Oncol 6: in press.

Siegall, C. B. et al. 1990. Cancer Research 50: 7786-7788.

Soule H. D. et al. 1973. J Natl Cancer Inst 51: 1409-1413.

Stampfer, M. R and Bartley, J. C. (1985) Proc. Natl. Acad. Sci. USA, 82: 2394-2398.

Stampfer, M. R. and Yaswen, P. 1993. Cancer Surveys 18: 7.

Tang, R. et al. 1990. J Cell Biochem. 44: 189.

Tanigawa, T., Nicola, N., McArthur, G. A., Strasser, A. and Begley, C. G. (1995) Blood, 85(2): 379-390.

Tannock I. et al. 1988. J Clin Oncol 6: 1377-1387.

Todd N. W. et al. 1993. Am Rev Respir Dis 147: 1264-1270.

Trempe G. and Fogh J. 1973. In vitro 8: 433.

Valagussa P., Bonadonna G., Veronesi U. 1978. Tumori 64: 241.

Van Oosterrom A. T. 1987. Clin Trials J 24: 131-137.

Walen K. and Stampfer M. R. 1989.Cancer Genetics and Cytogenetics 37: 249-261.

Wong G. et al. 1988. Behring Inst Mitt 83: 40-47.

Wood W. C. et al. 1994. N Eng J Med 330: 1253-1259.

Yamasaki K. et al. 1988. Science 241: 825-828.

Zhang, X., Gu, J., Lu, Z., Yasukawa, K., Yancopoulos, G. D., Tumer, K., Shoyab, M., Taga, T., (v/v) BCS, T., Bataille, R and Klein, B. (1994) J. Exp. Med., 177: 1337-1342.

1. A method for the treatment of prophylaxis breast cancer in a mammal, said method comprising administering to said mammal an effective amount of one or more cytokines or functional derivatives or agonists of said one or more cytokines for a time and under conditions sufficient to ameliorate the effects of or to delay onset of said cancer.

2. A method according to claim 1 wherein the cytokines are selected from the list consisting of oncostatin M (OSM), interleukin-6 (IL-6), interleukin-11 (IL-11), leukaemia inhibitory factor (LIF) and epidermal growth factor (EGF) or other members of the EGF family.

3. A method according to claim 1 wherein the cytokine is OSM.

4. A method according to claim 1 wherein the cytokine is LIF.

5. A method according to claim 2 or 3 or 4 wherein the cytokine is of human or murine origin.

6. A method according to claim 1 or 2 wherein the breast cancer is metastic breast cancer.

7. A method according to claim 1 or 2 wherein the breast cancer is early breast cancer.

8. A method according to any one of the preceding claims further comprising the simultaneous or sequential administration of at least one other therapeutic agent.

9. A method according to claim 8 wherein the other therapeutic agent is a chemotherapeutic agent or a hormone.

10. A method according to claim 9 wherein the chemotherapeutic agent is selected from cyclophosphamide, vincristine, methotrexate, cisplatin, melphalan and adriamycin.

11. A method according to claim 9 wherein the hormone is tamoxifen or other anti-estrogens.

12. A method according to claim 1 wherein the cytokine is administered in an amount from about 0.5 μg to about 2 mg per kilogram of body weight of mammal.

13. A method according to claim 1 wherein the mammal is a human.

14. A pharmaceutical composition effective in ameliorating the effects of breast cancer or delaying onset of breast cancer in a mammal comprising at least one cytokine or a functional derivative or agonist thereof and one or more pharmaceutically acceptable carriers and/or diluents.

15. A pharmaceutical composition according to claim 14 comprising a cytokine selected from IL-6, OSM, IL-11, LIF and EGF or other members of the EGF family.

16. A pharmaceutical composition according to claim 14 where the cytokine is OSM.

17. A pharmaceutical composition according to claim 14 wherein the cytokine is LIF.

18. A pharmaceutical composition according to claim 14 or 15 or 16 further comprising a chemotherapeutic agent or a hormone.

19. A pharmaceutical composition according to claim 18 wherein the chemotherapeutic agent is selected from cyclophosphamide, vincristine, methotrexate, cisplatin, melphalan and adriamycin.

20. A pharmaceutical composition according to claim 18 wherein the hormone is tamoxifen or other anti-estrogens.

21. Use of a cytokine or a functional derivative or agonist thereof in the manufacture of a medicament for the treatment or prophylaxis of breast cancer in a mammal.

22. Use according to claim 21 wherein the cytokine is IL-6, OSM, EGF, IL-11 or LIF.

23. Use according to claim 22 where the cytokine is OSM.

24. Use according to claim 22 wherein the cytokine is LIF.

25. Use according to claim 22 wherein the mammal is a human.

26. An agent comprising a cytokine or a functional derivative or agonist thereof for the treatment or prophylaxis of breast cancer in a mammal.

27. An agent according to claim 26 wherein the cytokine is IL-6, OSM, IL-11, LIF or EGF or other members of the EGF family.

28. An agent according to claim 26 wherein the cytokine is OSM

29. An agent according to claim 26 wherein the cytokine is LIF.
